(19) United States
(12) Patent Application Publication
(10) Pub. No.: US 2009/0325225 A1
(43) Pub. Date: Dec. 31, 2009

(54) METHOD FOR THE PRODUCTION OF (4S)-3,4-DIHYDROXY-2,6,6-TRIMETHYL-CYCLOHEX-2-ENONE AND DERIVATIVES THEREOF

(75) Inventors: Michael Breuer, Darmstadt (DE); Hansgeorg Ernst, Speyer (DE); Bernhard Hauer, Fussgonheim (DE)

Correspondence Address: CONNOLLY BOVE LODGE & HUTZ, LLP, P O BOX 2207, WILMINGTON, DE, 19899, US

(73) Assignee: BASF Aktiengesellschaft Patents, Trademarks and Licenses, Ludwigshafen (DE)

(21) Appl. No.: 12/514,188

(22) PCT Filed: Nov. 9, 2007
(86) PCT No.: PCT/EP2007/062170

§ 371 Date: May 8, 2009

(30) Foreign Application Priority Data
Nov. 10, 2006 (EP) 06123814.3

(51) Int. Cl. C12P 23/00

(52) U.S. Cl. 435/67

(57) ABSTRACT

The present invention relates to a method for preparing optically active (4S)-4-hydroxy-2,6,6-trimethylcyclohex-2-en-1-one derivatives of the formula (I) and a method for preparing (3S,3′S)-astaxanthin of the formula (III) comprising the first-mentioned method.

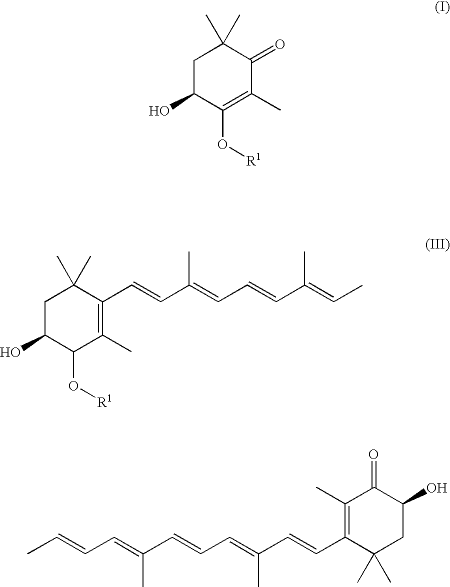

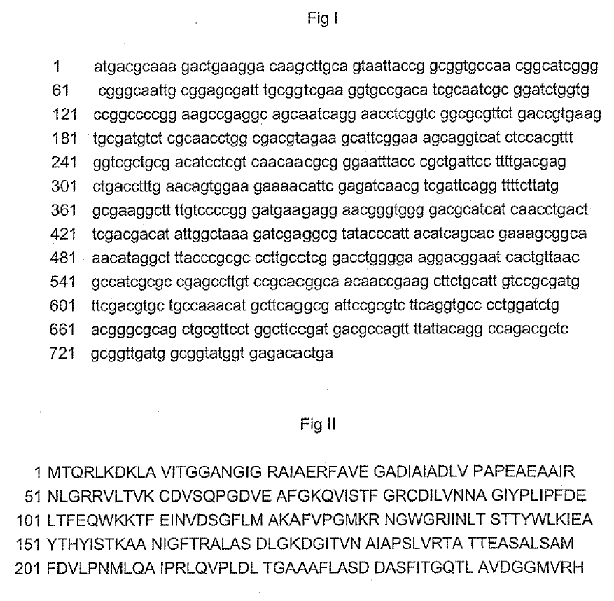

The present invention relates to a method for preparing optically active (4S)-4-hydroxy-2,6,6-trimethylcyclohex-2-en-1-one derivatives of the formula (I) and a method for preparing (3S,3′S)-astaxanthin of the formula (III) comprising the first-mentioned method.

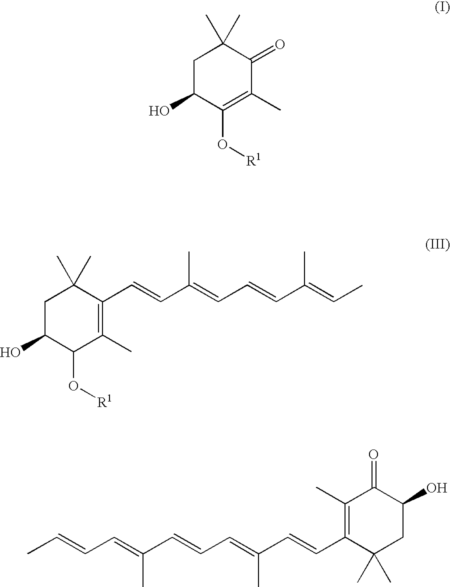

Because of its two chirality centers in position 3 and 3′, astaxanthin (3,3′-dihydroxy-β,β′-carotene-4,4′-dione) may exist in the form of the following configurational isomers: (3S,3′S), (3R,3′R), (3S,3′R) and (3R,3′S). The two last-mentioned configurational isomers are identical and represent a mesoform (Carotenoids Handbook, 2004, Main List No. 405).

All three forms are found in natural sources (Carotenoids Handbook, 2004, Main List No. 404, 405, 406). Total chemical synthesis starting from racemic precursors leads to a 1:2:1 mixture of (3S,3′S)-, meso- and (3R,3′R)-astaxanthih (The EFSA Journal, 2005, 291, 12, Deutsche Lebensmittel-Rundschau, 2004, 100, 437, Heivetica Chimica Acta, 1981, 64, 2436).

However, the (3S,3′S) configurational isomer is of particular importance. It is biosynthesized by green algae (Haematococcus pluvialis) in enantiopure form (J. Applied Phycology, 1992, 4, 165; Phytochemistry, 1981, 20, 2561).

(S,S)-Astaxanthin from green algae is employed as dietary supplement with beneficial effects for human health (J. Nat. Prod., 2006, 69, 443). It is additionally suitable for completely blocking the disadvantageous pro-oxidant effects of rofecoxib (Vioxx) (J. Cardiovasc. Pharmacol., 2006, 47 Suppl 1, S. 7).

Figures

In view of the low concentration of (S,S)-astaxanthin in green algae (J. Agric. Food Chem., 1998, 46, 3371), the availability of this active substance is, however, very limited. In addition, the active substance is present in the algae in a mixture of mono- and di-fatty acid esters plus free astaxanthin, causing considerable complexity in the isolation and purification (see inter alia Phytochemistry, 20, 11, 2561 (1981); J.Applied Phycology, 4, 2, 165 (1992)). In order to be able to provide (S,S)-astaxanthin in larger quantity and high purity, total chemical synthesis is the technology of choice.

Various syntheses of (S,S)-astaxanthin have been described in the literature. One strategy consists of resolving racemic precursors into the optical antipodes using diastereomeric salts (Helvetica Chimica Acta, 1981, 64, 2447) or diastereomeric esters (Helvetica Chimica Acta, 1981, 64, 2419). The microbial racemate resolution of racemic precursors has also been reported. A particular disadvantage of these methods is that the enantiomer which would lead to (R,R)-astaxanthin is not utilizable and can be recycled only with great complexity.

Another synthetic strategy consists of obtaining enantiopure synthesis building blocks by microbial or enzymatic methods (Helvetica Chimica Acta, 1978, 61, 2609, Helvetica Chimica Acta, 1981, 64, 2405). Since the oxidation state of these building blocks is too low, it was thus necessary to convert them in multistage syntheses into (S,S)-astaxanthin precursors.

WO 2006/039685 describes firstly in scheme II a two-stage enantioselective hydrogenation of ketoisophorone to give an enantiopure C9 diol, from which (S,S)-astaxanthin precursors are obtained in a multistage synthesis after reoxidation of a hydroxy group based on the method described in Helv.Chim.Acta, 1978, 61, 2609, WO 2006/039685 further describes an enantioselective catalytic transfer hydrogenation of a C9 enol ether of the formula (IIa) to give the corresponding enantiopure alcohol of the formula (Ia).

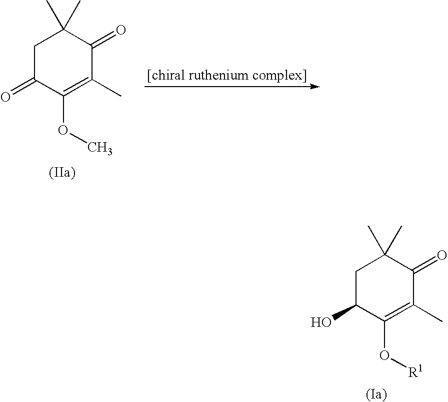

Hydrogenation catalysts which are described are metals with chiral ligands, preferably ruthenium catalysts with optically active amines as ligands. One disadvantage of this method is that an O-protected derivative of an industrial intermediate of the formula (IIb) is employed,

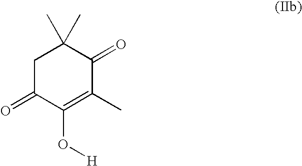

thus requiring additional synthetic effort. In addition, the required optically active catalysts are very costly, so that their use in industrial methods is impeded by economic considerations.

Biocatalytic reduction of the C₉ enol of the formula (IIb) with yeast have also been described. However, in this case an enantiomeric excess of only 65% was obtained, making this process of no use for an industrial method (Helv.Chim.Acta, 1981, 64, 2447).

It is known in principle that dehydrogenases are suitable as biocatalysts for preparing optically active hydroxy compounds. They are well-characterized biocatalysts which are already employed in a number of industrial processes (Angew. Chem. Int. Ed., 2004, 43, 788, Tetrahedron, 2004, 60, 633, Chiral catalysis—asymmetric hydrogenation supplement to Chemistry Today, 2004, 22, 26, Current Opinion in Chemical Biology, 2004, 8, 120, Organic Process Research & Development, 2002, 6, 558, Tetrahedron: Asymmetry, 2003, 14, 2659, Chiral catalysis—asymmetric hydrogenation supplement to Chemistry Today, 2004, 22, 43).

WO 2005/108590 describes a method for preparing certain optically active alkanols such as, for example, (1S)-3-methylamino-1-(2-thienyl)propan-1-ol or (1S)-3-chloro-1-(2-thienyl)propan-1-ol by enzymatic reduction of the corresponding ketones. The extent to which the enzymes used can be used to reduce ketones differing in structure is not discussed.

The described methods for introducing the two chirality centers into one of the precursors for synthesizing S,S′-astaxanthin are either complicated and multistage or uneconomic tasks so that these methods appear to be little suited for implementation on the industrial scale.

It was an object of the present invention to develop a simple and economically efficient method, starting from industrially available starting materials, for preparing an optically active intermediate, if possible in enantiopure form, for synthesizing (S,S′)-astaxanthin which can be integrated without difficulty into existing industrial total syntheses of “racemic” astaxanthin (Carotenoids Vol. 2, 1996, 259; Pure and Applied Chemistry, 2002, 74, 2213).

This subject is achieved by a method for preparing optically active (4S)-4-hydroxy-2,6,6-trimethylcyclohex-2-en-1-one derivatives of the formula (I)

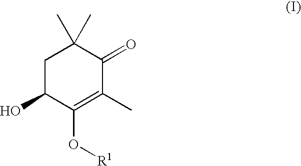

in which
R¹ is hydrogen, C₁-C₁₀-alkyl, C₇-C₁₄-arylalkyl, an alkali metal M¹ or an alkaline earth metal fragment M²_{1/2} or (M²)⁺X⁻, where M¹ is Li, Na, K, Rb or Cs and M² is Mg, Ca, Sr or Ba, and X⁻ is a singly charged anion,
comprising a reaction step
where an enzyme (E) selected from the class of oxidoreductases is incubated in a medium which comprises a trimethylcyclohex-2-ene-1,4-dione derivative of the formula (II)

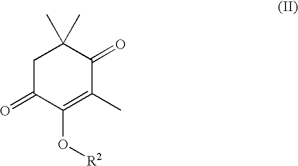

in which R² is identical to or different from R¹ and is hydrogen, C₁-C₁₀-alkyl, C₇-C₁₄-arylalkyl, an alkali metal M¹ or an alkaline earth metal fragment M²_{1/2} or (M²)⁺X⁻, where M¹ is Li, Na, K, Rb or Cs and M² is Mg, Ca, Sr or Ba, and X⁻ is a singly charged anion,
in the presence of reducing equivalents, with the compound of the formula (II) being enzymatically reduced to the compound of the formula (I), and the reducing equivalents consumed during the course of the reaction are regenerated again by converting a reducing means (RM) into the corresponding oxidation product (OP) with the aid of the enzyme (E) or of a further enzyme (E²), and optionally the oxidation product (OP) is at least partially removed from the reaction medium or from the reaction equilibrium, and the product (I) which is formed is isolated.

In the method according to the invention there is preparation of compounds of the formula (I) in which R¹ is hydrogen, C₁-C₁₀-alkyl such as, for example, methyl, ethyl, n-propyl, isopropyl or n-hexyl, C₇-C₁₄-arylalkyl such as, for example, benzyl, an alkali metal M¹ or an alkaline earth metal fragment M²_{1/2} or (M²)⁺X⁻, where M¹ is Li, Na, K, Rb or Cs, preferably Na or K, in particular Na, and M² is Mg, Ca, Sr or Ba, in particular Mg, and X⁻ is a singly charged anion such as, for example, halide, acetate or dihydrogen phosphate. R¹ is preferably hydrogen, methyl, Na or K, particularly preferably hydrogen, methyl or Na, especially hydrogen or sodium.

In the starting compounds of the formula (II) which are reacted in the method according to the invention, the radical R² is identical to or different from R¹ and is hydrogen, C₁-C₁₀-alkyl such as, for example, methyl, ethyl, n-propyl, isopropyl or n-hexyl, C₇-C₁₄-arylalkyl such as, for example, benzyl, an alkali metal M¹ or an alkaline earth metal fragment M²_{1/2} or (M²)⁺X⁻, where M¹ is Li, Na, K, Rb or Cs, preferably Na or K, in particular Na, and M² is Mg, Ca, Sr or Ba, in particular Mg, and X⁻ is a singly charged anion such as, for example, halide, acetate or dihydrogen phosphate. R¹ is preferably hydrogen, methyl, Na or K, particularly preferably hydrogen, methyl or Na, especially hydrogen or sodium.

Enzymes (E) of the class of oxidoreductases, meaning in particular enzymes having dehydrogenase activity, which are suitable according to the invention are in particular the enzymes of the families of aldo-keto reductases of the aldo-keto reductase superfamily (K. M. Bohren, B. Bullock, B. Wermuth und K. H. Gabbay J. Biol. Chem. 1989, 264, 9547-9551) and of the short-chain alcohol dehydrogenases/reductases (SDR). The latter enzyme group is for example described in detail in H. Jörnvall, B. Persson, M. Krook, S. Atrian, R. Gonzalez-Duarte, J. Jeffery and D. Ghosh, Biochemistry, 1995, 34, pp. 6003-6013 or U. Oppermann, C. Filling, M. Hult, N. Shafqat, X. Q. Wu, M. Lindh, J. Shafqat, E. Nordling, Y. Kallberg, B. Persson und H. Jornvall, Chemico-Biological Interactions, 2003, 143, pp. 247-253.

Within these enzyme classes mentioned, the alcohol dehydrogenases, especially the short-chain alcohol dehydrogenases, are especially well suited. Enzymes preferred among the alcohol dehydrogenases are in particular those which reduce, with NADH or NADPH as reducing equivalents, the compound of the formula (II) to the compound of the formula (I).

A particularly suitable embodiment of the method according to the invention consists of the enzyme (E) having a polypeptide sequence which either
(i) is SEQ ID NO: 2, or
(ii) in which up to 25% of the amino acid residues are altered by comparison with SEQ ID NO:2 by deletion, insertion, substitution or a combination thereof and which still has at least 50% of the enzymatic activity of SEQ ID NO:2.

Suitable enzymes (E) having oxidoreductase activity, especially dehydrogenase activity, which comprise an amino acid sequence as shown in SEQ ID NO: 2, and “functional equivalents” or analogues of the specifically disclosed enzymes (E) having oxidoreductase activity, especially dehydrogenase activity, which can likewise be employed in the method according to the invention, are described in detail in WO 2005/108590, pages 11 to 16, which is incorporated herein by reference.

An enzyme (E) having oxidoreductase activity, in particular dehydrogenase activity, which is preferably used in the method according to the invention can be prepared from microorganisms of the genera Azoarous, Azonexus, Azospira, Azovibrio, Dechloromones, Ferritacterium, Petrobacter, Propionivltrio, Quadricoccus, Rhodocyclus, Sterolibacterium, Thauera and Zoogloea.

An enzyme (E) having oxidoreductase activity, especially dehydrogenase activity, which is particularly preferably used in the method according to the invention is selected from enzymes from microorganisms of the genus Azoarcus, especially from the bacterium Azoarcus sp.EbN1.

On the basis of its amino acid sequence, the phenylethanol dehydrogenase from Azoarcus sp EbN1 can be included among the short-chain alcohol dehydrogenases reductases (SDR). The group of enzymes is described in detail for example in H. Jörnvall, B. Persson, M. Krook, S. Atrian, R. Gonzalez-Duarte, J. Jeffery and D. Ghosh, Biochemistry, 1995, 34, pp. 6003-6013 or U. Oppermann, C. Filling, M. Hult, N. Shafqat, X. Q. Wu, M. Lindh, J. Shafqat, E. Nordling, Y. Kallberg, B. Persson and H. Jornvall, Chemico-Biological Interactions, 2003, 143, pp. 247-253. The amino acid sequences of the individual representatives within the SDR group may differ widely from one another. It is nevertheless known that certain amino acids or amino acid regions are highly conserved within the SDR group. Important conserved regions of the SDR are described in C. Filling, K. D. Berndt, J. Benach, S. Knapp, T. Prozorovski, E. Nordling, R. Ladenstein, H. Jornvall and U. Oppermann, Journal of Biological Chemistry, 2002, 277, pp. 25677-25684.

Examples of Azoarcus species are Azoarcus, anaerobius, Azoarcus buckeil, Azoarcus communeis, Azoarcus evansii, Azoarcus indigens, Azoarcus toluclasticus, Azoarcus toluljticus, Azoarcus toluvorans, Azoarcus sp., Azoarcus sp. 22 Lin, Azoarcus sp. BH72, Azoarcusasp. CC-11, Azoarcus sp. CIB, Azoarcus sp. CR23, Azoarcus sp. EB1, Azoarcus sp. EbN1, Azoarcus sp. FLOS, Azoarcus sp. HA, Azoarcus sp. HxN1, Azoarcus sp. mXyN1, Azoarcus sp. PbN1, Azoarcus sp. PH002, Azoarcus sp. T and Azoarcus sp. ToN1.

Dehydrogenases from the bacterium Azoarcus sp. EbN1 are particularly preferably used as enzyme (E) in the method according to the invention.

The method according to the invention is preferably carried out in the presence of an enzyme (E) where the enzyme is encoded by a nucleic acid sequence as shown in SEQ ID NO:1 or a functional equivalent thereof.

The nucleic acid sequences which can be employed for encoding the enzymes (E) having dehydrogenase activity which can be used in the method according to the invention are described in detail in WO 20051108590, pages 16 to 22, which is incorporated herein by reference.

In a particularly preferred embodiment of the method, the enzyme having dehydrogenase activity is selected from enzymes which comprise an amino acid sequence as shown in SEQ ID NO: 2 or a sequence derived therefrom in which up to 25%, preferably up to 20%, particularly preferably up to 15%, in particular up to 10, 9, 8, 7, 6, 5, 4, 3, 2, 1% of the amino acid residues have been altered by a deletion, a substitution, an insertion or a combination of deletion, substitution and insertion, where the polypeptide sequences which are altered by comparison with SEQ ID NO: 2 still had at least 50%, preferably at least 65%, particularly preferably 80%, especially more than 90% of the enzymatic activity of SEQ ID NO:2. In this connection, enzymatic activity of SEQ ID NO:2 is intended to mean the ability to reduce ketones of the formula (II), especially with R¹═H or Na, enantioselectively to the (S) alcohol having the general formula (I).

The method according to the invention is carried out with addition of reducing equivalents, especially of NADH or NADPH, which serve as hydride source. Since NADH and NADPH are very costly compounds, these reducing equivalents are normally employed only in catalytic quantities. Reducing means (RM) which can be employed in principle for regenerating the reducing equivalents consumed in the reaction are inorganic or organic compounds such as, for example, phosphites or alcohols or else electrochemical methods such as reduction at a cathode. The reducing means (RM) preferably employed in the method according to the invention is an organic compound which comprises at least one primary or secondary alcohol functional CH(OH), such as, for example, isopropanol, 2-butanol, 2-pentanol, 2-hexanol, 3-hexanol or reducing sugars such as glucose, especially isopropanol or glucose. The reducing means (RM) is converted with the aid of the enzyme (E) or of a further enzyme (E²) into the oxidation product (OP), with the oxidation product (OP) being at least partially removed from the reaction medium or from the reaction equilibrium. If the reducing means (RM) comprises a secondary alcohol, it is also frequently referred to as sacrificial alcohol and the correspondingly formed oxidation product (OP) as sacrificial ketone.

In a preferred embodiment of the invention, the added sacrificial alcohol is employed not only for regenerating the consumed reducing equivalents but also as cosolvent. It is possible to operate in a liquid 1-phase, 2-phase or else multiphase system, one phase normally consisting of water and/or a water-miscible solvent.

The reducing equivalents are preferably employed in an amount of from 0.001 to 100 mmol, particularly preferably from 0.01 to 1 mmol, of reducing equivalents per mole of trimethylcyclohex-2-ene-1,4-dione derivative of the formula (II) employed.

The oxidation product (OP) formed in the method according to the invention can be at least partially removed from the reaction medium or from the reaction equilibrium. In the case of secondary alcohols such as isopropanol as reducing means (RM), it is possible to remove the so-called sacrificial ketones formed, acetone in the case of isopropanol as sacrificial alcohol, in various ways, for example through selective membranes or by extraction or distillation methods. Distillation is preferably employed to remove a ketone such as acetone. In the removal of the ketone by distillation there is normally also removal of part of the sacrificial alcohol and, in some cases, also parts of the aqueous phase. These distillation losses are ordinarily compensated by subsequent metering in of the sacrificial alcohol and, if appropriate, of the water. The distillation rates are normally in a range from 0.02% /min to 2%/min, preferably from 0.05%/min to 1%/min based on the reaction volume. The jacket temperatures of the reactor are between 5-70 kelvin, preferably between 10-40 kelvin, above the internal temperature of the reaction.

In the case of an involatile oxidation product (OP), as in the case of oxidation of glucose to gluconic acid, the latter is removed from the reaction equilibrium between reducing means (RM) and oxidizing means (OM) by cyclization to gluconolactone, Whereas oxidation of many sacrificial alcohols is possible by the same enzyme (E) which also catalyzes the reduction of compounds of the formula (II) to compounds of the formula (I), it is necessary to add a second enzyme (E²) such as, for example, glucose dehydrogenase for the oxidation of glucose.

In the case of volatile oxidation products, the distillation is carried out especially well in a pressure range of 1-500 mbar, preferably 10-200 mbar.

A preferred embodiment of the method according to the invention is for the conversion of the compound of the formula (II) into the compound of the formula (I) to take place in the presence of a microorganism which is selected from bacteria of the families Enterobacteriaceae, Pseudomonadaceae, Rhizobiaceae, Lactobacillaceae, Streptomycetaceae, Rhodococcaceae, Rhodocyclaceae and Nocardiaceae. The microorganism may be in particular a recombinant microorganism which is transferred with a nucleic acid construct which codes for an enzyme having oxidoreductase activity, preferably dehydrogenase activity, especially alcohol dehydrogenase activity as defined above. Expression constructs which comprise, under the genetic control of regulatory nucleic acid sequences, a nucleic acid sequence which codes for a protein which can be used in the method according to the invention, i.e. for an enzyme (E), and corresponding vectors which comprise at least one of the expression constructs are described in detail in WO 2005/108590, pages 22 to 25, which is incorporated herein by reference.

Recombinant microorganisms transformed with a suitable vector or construct and employable for producing the polypeptides which can be used in the method according to the invention, i.e. an enzyme (E), are described in detail in WO 2005/108590, pages 25 to 27, which is incorporated herein by reference.

Methods for the recombinant preparation of polypeptides or functional biologically active fragments thereof which comply with the function of the enzyme (E) in the method according to the invention, where a polypeptide-producing microorganism is cultured, expression of the polypeptides is induced if appropriate, and they are isolated from the culture, are described in detail in WO 2005/108590, pages 27 to 29, which is incorporated herein by reference. The polypeptides can also be produced on the industrial scale by the stated methods.

The enzymes (E) having oxidoreductase activity, especially dehydrogenase activity, used according to the invention can be used as free or immobilized enzyme (E) in the method according to the invention.

A preferred embodiment of the method according to the invention comprises at least a), b) and d) of the steps mentioned below:
a) to isolate from a natural source, or produce recombinantly, a microorganism producing an enzyme having dehydrogenase activity,
b) to propagate this microorganism,
c) to isolate the enzyme having dehydrogenase activity if appropriate from the microorganism, or to prepare a protein fraction comprising this enzyme, and
d) to transfer the microorganism from stage b) or the enzyme from stage c) into a medium which comprises a compound of the formula I.

The method according to the invention is advantageously carried out at a temperature between 0° C. and 95° C., preferably between 10° C. and 85° C., particularly preferably between 15° C. and 75° C.

The pH in the method according to the invention is advantageously kept at between pH 4 and 12, preferably between pH 4.5 and 9, particularly preferably between pH 5 and 8.

Enantiopure or chiral products or optically active alcohols mean in the method according to the invention enantiomers which show an enantiomeric enrichment. The method according to the invention achieves preferably enantiopurities of at least 70% ee, preferably of min. 80% ee, particularly preferably of min. 90% ee, very particularly preferably min. 98% ee.

It is possible to use for the method according to the invention growing cells which comprise suitable nucleic acids, nucleic acid constructs or vectors. Resting or disrupted cells can also be used. Disrupted cells mean for example cells which have been made permeable by a treatment with, for example, solvents, or cells which have been disintegrated by an enzymic treatment, by a mechanical treatment (e.g. French Press or ultrasound) or by another method. The crude extracts obtained in this way are advantageously suitable for the method according to the invention. Purified or partly purified enzymes (E) can also be used for the method. Likewise suitable are immobilized microorganisms or enzymes which can be advantageously used in the reaction.

If free organisms or enzymes are used for the method according to the invention, they are expediently removed, for example by filtration or centrifugation, before the extraction.

The compounds of the formula (I) prepared in the method according to the invention, for example (3S)-3,4-dihydroxy-2,6,6-trimethylcyclohex-2-enone can advantageously be isolated from the aqueous reaction solution by extraction or precipitation. The product solution is advantageously initially filtered to remove undissolved biological material, preferably with the addition of a filtration aid such as Celite.

The product (with R₁=alkyl) is then removed by extraction with a water-immiscible organic solvent. Examples of suitable solvents are toluene or other cyclic or open-chain hydrocarbons, chlorinated hydrocarbons such as, for example, methylene chloride, ethyl acetate or butyl acetate, and ethers such as MTBE or diisopropyl ether.

The compound of the formula I (with R¹═H or alkali metal or alkaline earth metal) can in principle be isolated from the reaction mixture as described in Helv.Chim.Acta 64, 2436, 1981. The product solution is initially adjusted to a pH of from 1 to 3, preferably pH 1. The acidification is preferably carried out with mineral acids such as, for instance, hydrochloric or sulfuric acid, particularly preferably with sulfuric acid. The product precipitates in this case and can be removed. However, the acidid product solution is preferably extracted several times with an organic solvent. Suitable solvents for this are chlorinated hydrocarbons, especially methylene chloride, ethers such as, for instance, MTBE or diisopropyl ether, and ethyl acetate. This extraction can be carried out batchwise or continuously. Extraction of the product can be assisted by concentrating the aqueous phase before the acidification or by “salting out”; however, these operations are not essential for removing the product from the reaction solution.

It is possible in said workup methods to isolate the product of the formula (I) of the method according to the invention in yields of from 60 to >95%, preferably from 80 to 95%, based on the substrate of the formula (II) employed for the reaction (such as, for example, with R²═Na). The product has a high enantiopurity of >98% ee. It can if desired be purified by crystallization as disclosed in Helv. Chim. Acta 64, 2436, 1981, but is preferably employed without further purification operation in the further synthesis of S,S-astaxanthin as disclosed in Helv. Chim. Acta 64, 2447, 1981.

The method according to the invention can be operated batchwise, semibatchwise or continuously.

The method according to the invention can be carried out advantageously in bioreactors as described for example in Biotechnology, Volume 3, 2nd Edition, Rehm et al. editors, (1993), especially Chapter II.

The present invention further relates to a method for preparing (3S,3′S)-astaxanthin of the formula (III) comprising the method described above for preparing optically active (4S)-4-hydroxy-2,6,6-trimethylcyclohex-2-en-1-one derivatives of the formula (I) as one reaction step of the overall synthesis of (3S5, 3′S)-astaxanthin. Both the synthetic steps for preparing the starting compounds of the formula (II) and the synthetic steps for converting the enantiopure compound of the formula (I) by a plurality of stages into (3S,3′S)-astaxanthin of the formula (III) are known in principle from the literature. Conversion of the optically pure compound of the formula (I) in which R¹ is hydrogen, and which has been obtained by having enantioselective reduction of (R²) in which R² is preferably Na, into (3S,3′S)-astaxanthin takes place without racemization as described variously in the literature (WO 2006/039685; Helv.Chim.Acta, 1981, 64, 2447; ibid., 1981, 64, 2405).

These methods correspond to industrial astaxanthin syntheses (Carotenoids, Vol. 2, 1996, 259; Pure and Appl.Chem., 2002, 74, 2213) and provide an industrially and economically advantageous access to (3S,3′S)-astaxanthin.

The present invention also relates to the use of an enzyme (E) having a polypeptide sequence which either
(i) is SEQ ID NO: 2, or
(ii) in which up to 25% of the amino acid residues are altered by comparison with SEQ ID NO:2 by deletion, insertion, substitution or a combination thereof and which still has at least 50% of the enzymatic activity of SEQ ID NO:2,
   for preparing compounds of the formula (I). The enzyme described above is preferably used for preparing compounds of the formula (I) in a method for preparing (3S,3′S)-astaxanthin, where the compound of the formula (I) is converted in further reaction steps into (3S,3′S)-astaxanthin of the formula (III).

The advantage of the method according to the invention is that compounds of the formula (I) are obtained with high enantiopurity associated with good yields of these compounds.

The invention is illustrated by the following examples which do not, however, restrict the invention.

### EXAMPLES

### Definitions

Compound 1: 2-Hydroxy-3,5,5-trimethylcyclohex-2-ene-1,4-dione (formula (II) with R² equal to hydrogen)
Compound 1a: Sodium 3,5,5-trimethyl-1,4-dioxocyclohex-2-en-2-olate (formula (II) with R² equal to sodium)
Compound 1b: 2-Methoxy-3,5,5-trimethylcyclohex-2-ene-1,4-dione (formula (II) with R² equal to methyl)
Compound 2: (4S)-3,4-Dihydroxy-2,6,6-trimethylcyclohex-2-enone (formula (I) with R¹ equal to hydrogen)
Compound 2b: (4S)-4-Hydroxy-3-methoxy-2,6,6-trimethylcyclohex-2-enone (formula (I) with R¹ equal to methyl)

### Example 1

### Preparation of Recombinant Phenylethanol Dehydrogenase

E. coli LU11558 prepared as described in WO 2005/108590, examples 1 and 2, was grown in 20 ml of LB-Amp/Spec/Cm (100 μg/l ampicillin; 100 μg/l spectinomycin; 20 μg/l chloramphenicol), 0.1 mM IPTG, 0.5 g/L rhamnose in 100 ml Erienmeyer flasks (baffles) at 37° C. for 18 h, centrifuged at 5000*g/10 min, washed once with 10 mM TRIS*HCl, pH 7.0, and resuspended in 2 ml of the same buffer.

Cell-free crude protein extract was prepared by disrupting E. coli LU11558 cell paste with 0.7 ml glass beads (d=0.5 mm) in a vibratory mill (3×5 min with intermediate cooling on ice).

### Example 2

### Determination of the Activity of the Recombinant Dehydrogenase from E. coli LU11558

10 μl of cell-free crude extract (example 1; approx. 10 mg/ml total protein) were incubated with shaking in a mixture of 770 μl of 50 mM K phosphate buffer (with 1 mM MgCl₂, pH 6.5), 100 μl of i-propanol, 100 μl of NADH solution (0.5M) and 20 μl of compound 1 (1M in DMSO). The mixtures were analyzed in analogy to example 3. On average, 0.13 mM 3,4-dihydroxy-2,6,6-trimethylcyclohex-2-enone were formed. In controlled experiments without addition of rhamnose in the culturing, no conversion was detectable.

### Example 3

### Analysis of Compounds 1 and 2

The precursor concentration and product concentration can be determined by HPLC. Besides the concentration it is also possible, depending on the choice of the stationary and mobile phase, to determine the ee.

| stationary phase: | Chiralpak AS-RH, 150 * 4.6 mm, Daicel, equilibrated | | | |
|---|---|---|---|---|
| | at 40° C. | | | |
| mobile phase: | Eluant A: 10 mM KH₂PO₄ | | | |
| | Eluant B: CH₃CN | | | |
| Gradient: | Time | A | B | Flow rates |
| | [min] | [%] | [%] | [ml/min] |
| | 0 | 90 | 10 | 0.5 |
| | 10 | 90 | 10 | 0.5 |
| | 11 | 60 | 40 | 0.5 |
| | 20 | 60 | 40 | 0.5 |

| | |
|---|---|
| Flow rate: | 0.5 ml/min |
| Detection: | UV detection at 260 nm |
| Retention times: | (+)-3,4-Dihydroxy-2,6,6-trimethylcyclohex-2-enone: |
| | approx. 9.3 min |
| | (−)-3,4-Dihydroxy-2,6,6-trimethyl-cyclohex-2-enone: |
| | approx. 9.8 min |
| | 2-Hydroxy-3,5,5-trimethyl-cyclohex-2-ene-1,4-dione: |
| | approx. 17.6 mm |

A calibration series is constructed with authentic material to allow the concentration of unknown samples to be determined and making assignment of the enantiomers possible.

### Example 4

### Preparation of Glucose Dehydrogenase for Cofactor Regeneration

Glucose dehydrogenase can be used for cofactor regeneration. The enzyme can be obtained commercially (e.g. Jülich Fine Chemicals Order No. 22.10 or 19.10) or from in-house sources. The latter comprises an E. coli XL10 Gold clone which comprises the glucose dehydrogenase gene from Bacillus subtilis (Genbank Acc. No. M12276) in the plasmid pUC19 (this construct is called E. coli LU11293).

The following medium was made up for fermentation of E. coli LU11293:

| | | |
|---|---|---|
| 560 | g | Yeast extract (65%) |
| 448 | g | Tryptone (Difco) |
| 42 | g | KH₂PO₄ |
| 84 | g | Na₂HPO₄ |
| 644 | g | Glycerol (99%) |
| 100 | mL | SL4 solution (5×) |
| 1 | g | Tegosipon 3062 |

Make up medium to 13.5 l with water, adjust pH to 7.0, remove about
300 ml for preculture, and then sterilize at 122° C. for 30 min.
Add sterile salt solution*
*Salt solution:
2.1 g CaCl₂ * 2 H₂O
3.5 g MgSO₄ * 7 H₂O
14 g NH₄Cl
14 ml Ampicillin solution (100 mg/ml)
Dissolve in 500 ml of water and sterilize by filtration.

150 ml portions of medium were sterilized in two 1 l Erlenmeyer flasks and completed with 5 ml of sterile salt solution. Inoculation from an LB-ampicillin agar plate was followed by incubation of the precultures at 37° C. and 200 rpm for 12 hours, and addition to the fermentation medium. The fermentation was started at 37° C., 0.1 bar internal pressure, pH 7.0 (controlled with 20% phosphoric acid and 25% NaOH) with a gasification rate of 7.5 l/min and 300 rpm (controlling pO₂ at between 20 and 50% with 10-20 l/min air feed and 500-1500 rpm). After 2 h, 0.1 mM IPTG was added for induction and, after a total of 13 h, the fermentation was terminated. The cells (1.3 kg) were harvested and washed and then stored at −20° C. until used (2-20 g/l in the mixture).

### Example 5

### Cofactor Regeneration

Regeneration of the cofactor can also be carried out by phenylethanol dehydrogenase. In this case, addition of a separate regenerating enzyme is unnecessary. Phenylethanol dehydrogenase accepts various simple alcohols as reducing means. They are oxidated to the corresponding carbonyl compounds. A simple alcohol suitable for regenerating NADH with phenylethanol dehydrogenase is isopropanol. If 10% isopropanol is used in the reaction mixture instead of glucose dehydrogenase and glucose, the activity of the phenylethanol dehydrogenase can be determined as shown in example 2.

### Example 6

### Preparation of (4S)-3,4-dihydroxy-2,6,6-trimethylcyclohex-2-enone (compound 2) using the recombinant phenylethanol dehydrogenase from Azoarcus sp. EbN1

E. coli LU11558 was grown, harvested and converted into cell-free crude extract as in example 1. This extract was mixed with 0.2 mM NAD+ and 5.4 ml of a 1.68 M sodium 3,5,5-trimethyl-1,4-dioxocyclohex-2-en-2-olate solution (compound 1a) and incubated at 30° C. for 48 h.

Cell-free crude extract from LU11558 (≈250 mg total protein)

| | | |
|---|---|---|
| 19.8 | g | Glucose |
| 0.072 | g | NAD, Na salt |

GDH crude extract (≈180 mg total protein)

| | | |
|---|---|---|
| 450 | ml | Buffer solution (50 mM K phosphate, 1 mM MgCl₂ pH 6.5) |
| 5.4 | ml | Sodium 2-hydroxy-3,5,5-trimethyl-4-oxocyclohex-2-enolate |
| | | solution (1.68M) |

5.4 ml of the substrate solution were added after 4.75 h, and 16.2 ml were added after 6 h. The reaction was kept at pH 6.0-7.0 by titration with 5 M NaOH and 1 M HCl and was followed by HPLC analysis.

### Example 7

### Preparation of (4S)-4-hydroxy-3-methoxy-2,6,6-trimethylcyclohex-2-enone using a recombinant dehydrogenase from Azoarcus sp. EbN1

E. coli LU11558 was grown, harvested and converted into cell-free crude extract as in example 1. This extract was mixed with 0.2 mM NAD+ and 5.4 ml of a 1.68 M sodium 3,5,5-trimethyl-1,4-dioxocyclohex-2-en-2-olate solution and incubated at 30° C. for 48 h.

Cell-free crude extract from E. coli
LU11558 (≈20 mg total protein)

| | | |
|---|---|---|
| 19.8 | g | Glucose |
| 0.014 | g | NAD, Na salt |
| 10 | ml | i-Propanol |
| 450 | ml | Buffer solution (50 mM K phosphate, 1 mM MgCl₂ pH 6.5) |
| 1.822 | g | 2-Methoxy-3,5,5-trimethylcyclohex-2-ene-1,4-dione |
| | | (compound 1b) |

The reaction was followed by HPLC analysis. After 7 hours, the amount of precursor was replenished by adding 3.6 g of 2-methoxy-3,5,5-trimethylcyclohex-2-ene-1,4-dione, More than 60% of the precursor was consumed after 75 hours.

### DESCRIPTION OF THE FIGURES

FIG. I depicts SEQ ID NO:1 which is the nucleic acid sequence of the phenylethanol dehydrogenase from Azoarcus sp EbN1 (Genbank ID 25956124, region:25073 to 25822).

FIG. II depicts SEQ ID NO:2 which is the amino acid sequence of the phenylethanol dehydrogenase from Azoarcus sp EbN1 (Genbank protein ID CAD58337).

1-13. (canceled)

14. A method for preparing optically active (4S)-4-hydroxy-2,6,6-trimethylcyclohex-2-en-1-one derivatives of formula (I) 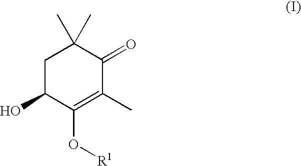
wherein
R¹ is hydrogen, C₁-C₁₀-alkyl, C₇-C₁₄-arylalkyl, an alkali metal M¹, or an alkaline earth metal fragment M²_{1/2} or (M²)⁺X⁻; wherein M¹ is Li, Na, K, Rb, or Cs and M² is Mg, Ca, Sr, or Ba, and X⁻ is a singly charged anion,
comprising
incubating an enzyme (E) selected from the class of oxidoreductases in a medium which comprises a trimethylcyclohex-2-ene-1,4-dione derivative of formula (II) 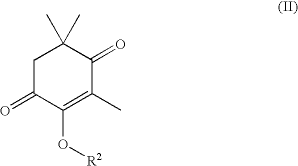
wherein R² is identical to or different from R¹ and is hydrogen, C₁-C₁₀-alkyl, C₇-C₁₄-arylalkyl, an alkali metal M¹, or an alkaline earth metal fragment M²_{1/2} or (M²)⁺X⁻, wherein M¹ is Li, Na, K, Rb, or Cs and M² is Mg, Ca, Sr, or Ba, and X⁻ is a singly charged anion,
in the presence of reducing equivalents,
wherein said compound of formula (II) is enzymatically reduced to said compound of formula (I),
wherein reducing equivalents consumed during the course of the reaction are regenerated again by converting a reducing means (KM) into the corresponding oxidation product (OP) with the aid of the enzyme (E) or of a further enzyme (E²), wherein the oxidation product (OP) is optionally at least partially removed from said medium or from the reaction equilibrium, and
wherein said compound of formula (I) which is formed is isolated.

15. The method of claim 14, wherein said enzyme (E) is an alcohol dehydrogenase.

16. The method of claim 15, wherein said alcohol dehydrogenase reduces, with NADH or NADPH as reducing equivalents, said compound of formula (II) to said compound of formula (I).

17. The method of claim 14, wherein said enzyme (E) has a polypeptide sequence
(i) which is SEQ ID NO: 2; or
(ii) wherein up to 25% of the amino acid residues are altered compared to SEQ ID NO:2 by deletion, insertion, substitution, or a combination thereof, and which still has at least 50% of the enzymatic activity of SEQ ID NO:2.

18. The method of claim 14, where said enzyme (E) is selected from enzymes from microorganisms of the genus Azoarcus.

19. The method of claim 18, wherein said enzyme (E) is selected from enzymes from the bacterium Azoarcus sp.EbN1.

20. The method of claim 14, where said enzyme (E) is encoded by a nucleic acid sequence as shown in SEQ ID NO:1 or a functional equivalent thereof.

21. The method of claim 14, wherein said reducing means (RM) comprises an organic compound comprising at least one primary or secondary alcohol function CH(OH).

22. The method of claim 21, wherein said organic compound comprising at least one primary or secondary alcohol function CH(OH) is isopropanol or glucose.

23. The method of claim 14, where the conversion of the compound of the formula (II) takes place in the presence of a microorganism which is selected from bacteria of the families Enterobacteriaceae, Pseudomonadaceae, Rbizobiaceae, Lactobacillaceae, Streptomycetaceae, Rhodococcaceae, Rhodocyclaceae and Nocardiaceac.

24. The method of claim 14, where the microorganism is a recombinant microorganism which is transformed with a nucleic acid construct which codes for an enzyme as defined in claim 14.

25. The method of claim 14, where R¹ in formula (I) is hydrogen, methyl or sodium, especially hydrogen or sodium.

26. A method for preparing (3S,3′S)-astaxanthin comprising the method of claim 14 as one reaction step in the overall synthesis of (3S,3′S)-astaxanthin.


SEQUENCE LISTING (2 sequences)

SEQ ID NO: 1 - DNA, 750 bp, Azoarcus sp.
atgacgcaaagactgaaggacaagcttgcagtaattaccggcggtgccaacggcatcgggcgggcaattgcggagcgatttgcggtcgaaggtgccgacatcgcaatcgcggatctggtgccggccccggaagccgaggcagcaatcaggaacctcggtcggcgcgttctgaccgtgaagtgcgatgtctcgcaacctggcgacgtagaagcattcggaaagcaggtcatctccacgtttggtcgctgcgacatcctcgtcaacaacgcgggaatttacccgctgattccttttgacgagctgacctttgaacagtggaagaaaacattcgagatcaacgtcgattcaggttttcttatggcgaaggcttttgtccccgggatgaagaggaacgggtggggacgcatcatcaacctgacttcgacgacatattggctaaagatcgaggcgtatacccattacatcagcacgaaagcggcaaacataggctttacccgcgcccttgcctcggacctggggaaggacggaatcactgttaacgccatcgcgccgagccttgtccgcacggcaacaaccgaagcttctgcattgtccgcgatgttcgacgtgctgccaaacatgcttcaggcgattccgcgtcttcaggtgcccctggatctgacgggcgcagctgcgttcctggcttccgatgacgccagttttattacaggccagacgctcgcggttgatggcggtatggtgagacactga

SEQ ID NO: 2 - protein, 249 aa, Azoarcus sp.
MTQRLKDKLAVITGGANGIGRAIAERFAVEGADIAIADLVPAPEAEAAIRNLGRRVLTVKCDVSQPGDVEAFGKQVISTFGRCDILVNNAGIYPLIPFDELTFEQWKKTFEINVDSGFLMAKAFVPGMKRNGWGRIINLTSTTYWLKIEAYTHYISTKAANIGFTRALASDLGKDGITVNAIAPSLVRTATTEASALSAMFDVLPNMLQAIPRLQVPLDLTGAAAFLASDDASFITGQTLAVDGGMVRH